SEQUENCE LISTING (15 sequences)

SEQ ID NO: 1 - DNA, 10 bp, Artificial Sequence
  Kozak translation initiation sequence
rccrccatgg

SEQ ID NO: 2 - DNA, 10 bp, Artificial Sequence
  Kozak translation initiation sequence
gccaccatgg

SEQ ID NO: 3 - DNA, 10 bp, Artificial Sequence
  Kozak translation initiation sequence
accaccatgg

SEQ ID NO: 4 - DNA, 34 bp, Artificial Sequence
  loxP recombination site sequence
ataacttcgtataatgtatgctatacgaagttat

SEQ ID NO: 5 - DNA, 32 bp, Artificial Sequence
  rox recombination site sequence
taactttaaataatgccaattatttaaagtta

SEQ ID NO: 6 - DNA, 34 bp, Artificial Sequence
  VloxP recombination site sequence
tcaatttctgagaatgacagttctcggaaattga

SEQ ID NO: 7 - DNA, 34 bp, Artificial Sequence
  SloxP recombination site sequence
ctcgtgtccgataactgtaattatcggacatgat

SEQ ID NO: 8 - DNA, 34 bp, Artificial Sequence
  vox recombination site sequence
aataggtctgagaatgggcgttctcagacgtatt

SEQ ID NO: 9 - DNA, 21 bp, Artificial Sequence
  attP recombination site sequence
cagcttttttatactaagttg

SEQ ID NO: 10 - DNA, 34 bp, Artificial Sequence
  FRT recombination site sequence
gaagttcctatactttctagagaataggaacttc

SEQ ID NO: 11 - DNA, 31 bp, Artificial Sequence
  RRT recombination site sequence
ttgatgaaagaataacgtattctttcatcaa

SEQ ID NO: 12 - DNA, 43 bp, Artificial Sequence
  KwRT recombination site sequence
acgaaaaatggtaaggaatagaccattccttaccatttttcgt

SEQ ID NO: 13 - DNA, 61 bp, Artificial Sequence
  KdRT recombination site sequence
aaacgatatcagacatttgtctgataatgcttcattatcagacaaatgtctgatatcgttt

SEQ ID NO: 14 - DNA, 39 bp, Artificial Sequence
  B2RT recombination site sequence
gagtttcattaaggaataactaattccctaatgaaactc

SEQ ID NO: 15 - DNA, 33 bp, Artificial Sequence
  B3RT recombination site sequence
ggttcgttaagaataagtaattcttaagcaacc

(19) United States
(12) Patent Application Publication
(10) Pub. No.: US 2019/0112600 A1
(43) Pub. Date: Apr. 18, 2019

(54) CONDITIONAL PROTEIN TRANSLATION SWITCHES, CONDITIONAL GENE EXPRESSION SYSTEMS AND USES THEREOF

(71) Applicant: Massachusetts Institute of Technology, Cambridge, MA (US)

(72) Inventor: Patrick J. Stern, Needham, MA (US)

(73) Assignee: Massachusetts Institute of Technology, Cambridge, MA (US)

(21) Appl. No.: 16/157,183

(22) Filed: Oct. 11, 2018

Related U.S. Application Data
(60) Provisional application No. 62/571,859, filed on Oct. 13, 2017.

(51) Int. Cl. C12N 15/11; C12N 15/85; C12N 15/63

(52) U.S. Cl. CPC C12N 15/111; C12N 15/85; C12N 15/635; C12N 2840/102; C12N 2320/30; C12N 2800/30; C12N 2830/50; C12N 2310/14

(57) ABSTRACT

Disclosed herein are protein translation switches and conditional gene expression systems that are compatible with retroviral and lentiviral gene delivery. The linking of a protein translation switch to a 3′ gene of interest suppresses translation of the gene of interest, and the alteration of the protein translation switch by DNA recombinase-mediated DNA recombination relieves the suppressed translation of the 3′ gene of interest. Also disclosed herein are methods of mimicking clinical pharmacology in a pre-clinical setting.

### RELATED APPLICATION

This application claims priority under 35 U.S.C. § 119(e) to U.S. provisional patent application No. 62/571,859, filed Oct. 13, 2017, the entire contents of which are incorporated herein by reference.

### GOVERNMENT SUPPORT

This invention was made with Government support under Grant Nos. U54 CA112967, ROI CA17007, and U54 CA126515 awarded by the National Institutes of Health. The Government has certain rights in the invention.

### FIELD

Disclosed herein are protein translation switches and conditional gene expression systems that are compatible with retroviral and lentiviral gene delivery. The linking of a protein translation switch to a 3′ gene of interest suppresses translation of the gene of interest, and the alteration of the protein translation switch by DNA recombinase-mediated DNA recombination relieves the suppressed translation of the 3′ gene of interest. Also disclosed herein are methods of mimicking clinical pharmacology in a pre-clinical setting.

### BACKGROUND

Conditional gene expression systems—which are encoded in genetic constructs—can facilitate expression-mediated regulation of a gene(s) of interest. Various conditional gene expression system designs that include enzyme-mediated DNA recombination have been demonstrated previously (Gierut J. J., et al., Cold Spring Harb Protoc. 2014 Apr. 1; 2014(4):339-49; Sacher T., et al., Med Microbiol Immunol. 2008 June; 197(2):269-76; Garcia-Otin A. L. and Guillou F., Front Biosci. 2006 Jan. 1; 11:1108-36); for example, conditional gene expression systems that utilize RNA processing elements (e.g., transcription suppressors, transcription terminators, or polyadenylation elements) and/or RNA translation suppressors composed of RNA sequences that form highly-stable, stem-looped RNA tertiary structures. Retroviral- and, in particular, lentiviral-based methodologies are ideal for delivering genetic constructs to diverse experimental and clinical cell types. However, many previously described conditional gene expression systems are incompatible with retroviral and/or lentiviral gene delivery methodologies. First, many known systems comprise 3-5 kb of DNA or more and are, thus, too large when combined with the viral genome to be delivered via retroviral and/or lentiviral methodologies. Second, because the viral genome is composed of RNA, foreign RNA processing elements and highly-structured RNA stem-loop intermediates disrupt the fidelity of the viral genome and the assembly of viral particles, thus perturbing the production of viable viruses.

### SUMMARY

Conditional gene expression systems can be a valuable part of the experimentalist's toolkit for dissecting biological processes. Application of these systems allows a biological process of interest to progress to a desired state (e.g., confluent growth in a dish or a mature tumor in a mouse), and then the process can be manipulated experimentally by “conditional” gene expression. Disclosed herein are conditional protein translation switches and conditional gene expression systems that are compatible with retroviral and lentiviral gene delivery. An ideal conditional gene expression system permits virtually no protein production of a gene(s) of interest in the OFF state and can then rapidly permit induced protein levels when induced to the ON state. The conditional gene expression systems described herein exhibit tight basal control over protein production of a gene(s) of interest and permit sub-minute time-scale protein expression kinetics. In one aspect, compositions of engineered conditional protein translation switches are disclosed. In some embodiments, a conditional protein translation switch comprises a polynucleic acid sequence comprising at least two Kozak translation initiation sequences together flanked by 5′ and 3′ recombination site sequences, which permit removal of the at least two Kozak translation initiation sequences from the polynucleic acid in the presence of a DNA recombinase that specifically recognizes the 5′ and 3′ recombination site sequences, and wherein: linking the protein translation switch to a sequence encoding a gene of interest placed 3′ to the 3′ recombination site sequence suppresses translation of the gene of interest, and the removal of the at least two Kozak translation initiation sequences from the conditional protein translation switch by DNA recombinase-mediated DNA recombination relieves the suppressed translation of the gene of interest. In some embodiments, the polynucleic acid sequence of the conditional protein translation switch comprises two or three Kozak translation initiation sequences together flanked by the 5′ and 3′ recombination site sequences. In some embodiments, the at least two Kozak translation initiation sequences of a protein translation switch are positioned 1, 2, or 3 nucleotides from each other. In some embodiments, the sequence of each of the at least two Kozak translation initiation sequences of a protein translation switch is RCCRCCATGG (SEQ ID NO: 1), with R being A or G. In some embodiments, the sequence of each of the at least two Kozak translation initiation sequences is GCCACCATGG (SEQ ID NO: 2). In some embodiments, the engineered conditional protein translation switch comprises recombination site sequences that are selected from the group consisting of loxP, rox, VloxP, SloxP, vox, attP, FRT, RRT, KwRT, KdRT, B2RT, and B3RT site sequences. In some embodiments, all of the recombination site sequences are loxP site sequences.

In another aspect, compositions of engineered conditional gene expression systems are provided. In some embodiments, the engineered conditional gene expression system comprises at least one polynucleic acid wherein the at least one polynucleic acid encodes at least one DNA recombinase and at least one conditional protein translation switch operably linked to a polynucleotide sequence encoding a gene of interest, wherein each conditional protein translation switch comprises a polynucleotide sequence comprising at least two Kozak translation initiation sequences together flanked by 5′ and 3′ recombination site sequences which permit removal of the at least two Kozak translation initiation sequences from the polynucleic acid in the presence of a DNA recombinase that specifically recognizes the 5′ and 3′ recombination site sequences, and each conditional protein translation switch is operably linked to the polynucleotide sequence encoding the gene of interest that is placed 3′ to the 3′ recombination site sequence, wherein the orientation of the at least one conditional protein translation switch relative to the gene of interest is such that the polynucleotide sequence encoding the gene of interest is out of translation reading frame with the at least two Kozak translation initiation sequences of the at least one conditional protein translation switch.

In some embodiments, the engineered conditional gene expression system comprises more than one protein translation switch, wherein each protein translation switch is operably linked to a polynucleotide sequence encoding a gene of interest, optionally wherein the polynucleotide sequences encoding a gene of interest are different sequences. In some embodiments, each protein translation switch comprises unique recombination site sequences.

In some embodiments, the engineered conditional gene expression system also comprises at least one protein-coding gene sequence, wherein each protein-coding gene sequence is positioned 3′ of the 5′ recombination site sequence and 5′ of the at least two Kozak translation initiation sequences of a conditional protein translation switch, and DNA recombinase-mediated DNA recombination at the recombination sites removes the protein-coding gene sequence and the at least two Kozak translation initiation sequences from the conditional protein translation switch. In some embodiments, each protein-coding sequence is uniquely positioned 3′ of the 5′ recombination site sequence and 5′ of the at least two Kozak translation initiation sequences of a conditional protein translation switch.

In some embodiments, the at least one DNA recombinase of a conditional gene expression system is selected from the group consisting of Cre, Dre, VCre, SCre, Vika, λ-Int, Flp, R, Kw, Kd, B2, B3, and functional variants thereof. In some embodiments, the at least one DNA recombinase is an inducible DNA recombinase. In some embodiments, the inducible DNA recombinase is selected from the group consisting of CreER and CreERT2.

In some embodiments, each conditional protein translation switch of a conditional gene expression system comprises a polynucleotide sequence comprising two or three Kozak translation initiation sequences. In some embodiments, the at least two Kozak translation initiation sequences of each conditional protein translation switch are positioned 1, 2, or 3 nucleotides from each other. In some embodiments, the sequence of at least one of the at least two Kozak translation initiation sequences of at least one conditional protein translation switch is RCCRCCATGG (SEQ ID NO: 1), wherein R is A or G. In some embodiments, the sequence of each Kozak translation initiation sequence of each conditional protein translation switch is RCCRCCATGG (SEQ ID NO: 1), wherein R is A or G. In some embodiments, the sequence of at least one Kozak translation initiation sequence of at least one conditional protein translation switch is GCCACCATGG (SEQ ID NO: 2). In some embodiments, each Kozak translation initiation sequence of each conditional protein translation switch is GCCACCATGG (SEQ ID NO: 2).

In some embodiments, the recombination site sequences of at least one conditional protein translation switch of a conditional gene expression system is selected from the group consisting of loxP, rox, VloxP, SloxP, vox, attP, FRT, RRT, KwRT, KdRT, B2RT, and B3RT site sequences. In other embodiments, the recombination site sequences of each conditional protein translation switch is selected from the group consisting of loxP, rox, VloxP, SloxP, vox, attP, FRT, RRT, KwRT, KdRT, B2RT, and B3RT site sequences. In some embodiments, the recombination site sequences of each conditional protein translation switches is a loxP site sequence.

In some embodiments, a single polynucleic acid encodes for the at least one DNA recombinase and the at least one conditional protein translation switch.

In another aspect, compositions of recombinant viral genomes are provided comprising any conditional gene expression system described above. In some embodiments, the viral genome is an adenovirus genome or a lentivirus genome.

In another aspect, compositions of engineered virions are provided comprising at least one conditional gene expression system described above. In some embodiments, the virion is an adenovirus virion or a lentivirus virion. In some embodiments, the engineered virion comprises the recombinant viral genome described above.

In another aspect, methods of testing the effect of a product of a gene of interest are provided. In some embodiments, the method of testing the effect of a product of a gene of interest comprises introducing a engineered conditional gene expression system (described above), a recombinant viral genome (described above), or an engineered virion (described above) into a biological sample, a cell or a test animal, expressing the at least one DNA recombinase or inducing the at least one inducible DNA recombinase to initiate DNA recombination, and analyzing the impact of DNA recombination relative to a control. In some embodiments, the gene of interest is identified by a gene deletion screen. In some embodiments, the cell is a tumor cell.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1B. Conditional gene expression systems used in this study. FIG. 1A. Schematic depicting the composition of a general conditional gene expression system and the mechanism whereby a protein translation switch facilitates the conditional translation of a gene of interest. The general conditional gene expression system used in these studies includes a polynucleic acid(s) that encode for 1) a protein translation switch—which includes DNA recombination site sequences that flank one or more Kozak translation initiation sequences—and 3′ a gene of interest and 2) a DNA recombinase (here encoded by a separate polynucleic acid). An optional protein-coding sequence can also be included in the conditional gene expression system (here represented by a selective drug). Enzyme-mediated, site-specific DNA recombination results in alteration of the protein translation switch (i.e., removal of the Kozak translation initiations sequences and any included optional protein-coding sequence). This recombination facilitates translation of the gene of interest and, thus, stimulates induction of the gene's protein levels. FIG. 1B. Schematics depicting polynucleic acid components of conditional gene expression systems used in this study: Flox-Puro^{R}/ZsGreen, which facilitates conditional translation of ZsGreen; Flox-Drug^{R}/Luciferase, which facilitates conditional translation of Luciferase; Flox-Neo^{R}/mCherry, which facilitates conditional translation of mCherry; Flox-AKT^{Wt}/AKT*, which facilitates conditional translation of AKT*, an active-site mutant kinase-dead AKT; Flox-Puro^{R}/TGFBRII-DN, which facilitates conditional translation of TGFBRII-DN (dominant negative TGF-β Receptor II), a cytoplasmic truncation that blocks downstream signaling after binding of TGF-β; and Flox-Puro^{R}/BH3, which facilitates conditional translation of BH3, a mitochondria-localized protein that promotes programmed cell death (apoptosis).

FIG. 2. The number of Kozak translation initiation sequences in a conditional translation suppressor switch, impacts the switch's efficiency. Translation “read through” re-initiation—which can lead to translation of the gene of interest (here ZsGreen)—decreases as the number of Kozak translation initiation sequences 5′ to a gene of interest increases. Translation suppressor switches with two and three Kozak sequences functioned similarly (i.e., tight restriction of ZsGreen production in the absence of Tamoxifen and robust production after cellular exposure to Tamoxifen). Detection of ZsGreen positive MEFs was assessed via FACs sorting.

FIGS. 3A-3E. The Flox-Puro^{R}/ZsGreen conditional gene expression system efficiently functions in vitro in various cell lines. Cells harboring the Flox-Puro^{R}/ZsGreen conditional gene expression system were analyzed by FACs sorting. Without exposure to Tamoxifen, ZsGreen positive cells were largely undetectable. However, cellular exposure to Tamoxifen resulted in a dramatic increase in the number of ZsGreen positive cells. FIG. 3A. 4T1 carcinoma cells. FIG. 3B. Immortalized MEF cells. FIG. 3C. B16 melanoma cells. FIG. 3D. D2.0R non-metastatic Balb/c cells. FIG. 3E. D2.A1 metastatic Balb/c cells.

FIGS. 4A-4B. Conditional gene expression systems remain efficient in cells harboring multiple expression systems. FIG. 4A. In the absence of Tamoxifen, ZsGreen positive (left box) and mCherry positive (right box) cells harboring the Flox-Puro^{R}/ZsGreen or the Flox-Puro^{R}/mCherry conditional gene expression systems, respectively, are largely undetectable via FACs sorting. However, following administration of Tamoxifen the number of ZsGreen positive (left box) and mCherry positive (right box) cells increases dramatically. FIG. 4B. In the absence of Tamoxifen, ZsGreen, mCherry positive cells harboring the Flox-Puro^{R}/ZsGreen and the Flox-Puro^{R}/mCherry conditional gene expression systems are largely undetectable via FACs sorting. However, following administration of Tamoxifen the number of ZsGreen, mCherry positive cells increases dramatically.

FIGS. 5A-5D. Conditional gene expression systems efficiently function in vivo. Lewis lung carcinoma cells (FIG. 5A) or primary mouse fibroblasts (FIG. 5B) harboring the Flox-Puro^{R}/Luciferase conditional gene expression system or 4T1 carcinoma cells harboring the Flox-Puro^{R}/ZsGreen conditional gene expression system were subcutaneously transplanted into the mouse fat pad of adult mice (FIG. 5D). FIG. 5A. The left image depicts Luciferase levels in two independent mice following cell transplantation (in the absence of Tamoxifen). Luciferase levels were largely undetectable. The right image depicts Luciferase levels, in the same mice 48 hours post Tamoxifen treatment. Robust luciferase levels were seen at the transplantation site. FIG. 5B. The top image depicts Luciferase levels in five independent mice 7 days after cell transplantation (in the absence of Tamoxifen). Luciferase levels were largely undetectable. The bottom image depicts Luciferase levels in the same mice 7 days post Tamoxifen treatment (i.e., 14 days post transplantation). Robust luciferase levels were seen at the transplantation site. FIG. 5C. Radiance scale for assessing Luciferase protein levels in FIG. 5A and FIG. 5B. FIG. 5D. The top image depicts ZsGreen levels in a 4T1 tumor (in the absence of Tamoxifen). ZsGreen levels were largely undetectable. The bottom image depicts ZsGreen levels in a 4T1 tumor 5 days post Tamoxifen treatment. Robust ZsGreen levels were seen throughout the tumor.

FIGS. 6A-6B. Kinetics of conditional gene expression systems. FIG. 6A. Recombination of the Puro^{R}/ZsGreen polynucleic acid sequence occurs within 1 minute of cellular exposure to Tamoxifen. Cells harboring the Puro^{R}/ZsGreen conditional gene expression system were exposed to Tamoxifen for the time lengths indicated on the X-axis. Following cellular exposure to Tamoxifen, the cells were washed to remove any residual Tamoxifen. Induction of ZsGreen protein levels were assessed 48 hours later by FACs sorting. FIG. 6B. Efficient translation of the gene of interest is rapidly induced following enzyme-mediated removal of the translational repressor. Induction of Luciferase protein levels was detectable within three hours following administration of Tamoxifen to cells harboring the Flox-Puro^{R}/Luciferase conditional gene expression system.

FIG. 7. The Flox-AKT^{Wt}/AKT* conditional gene expression system models clinical kinase inhibitor treatment in the pre-clinical setting. miRNA-mediated endogenous AKT knockdown was performed in MCF10A cells harboring the Flox-AKT^{Wt}/AKT* conditional gene expression system. Cells harboring the expression system were selected, plated, and grown for 2 days. Cell growth was assessed thereafter after treatment with Tamoxifen (AKT*) or absence of Tamoxifen (AKT^{Wt}). While cell growth was robust in the absence of Tamoxifen, administration of Tamoxifen resulted in rapid inhibition of cell growth.

FIG. 8. The Flox-Puro^{R}/TGFBRII-DN conditional gene expression system models clinical inhibition of TGF-β signaling in the pre-clinical setting. 4T1 carcinoma cells harboring the Flox-Puro^{R}/TGFBRII-DN conditional gene expression system or the Flox-Puro^{R}/ZsGreen conditional gene expression system were subcutaneously transplanted into adult mice and grown for 3 weeks. Tumor size was assessed after administration of Tamoxifen. Blockage of TGF-β resulted in rapid decreases in tumor size. While the tumor size in the ZsGreen samples continued to grow after treatment of Tamoxifen, tumor size in the TGFBRII-DN samples decreased rapidly.

FIGS. 9A-9E. The Flox-Puro^{R}/BH3 conditional gene expression system models clinical cytotoxic chemotherapy in the pre-clinical setting. The Flox-Puro^{R}/BH3 expression system allows for conditional expression of BH3, which drives apoptosis through mitochondrial disruption. Cells harboring this expression system mimic cytotoxic chemotherapy treatment in vitro and in vivo. While cell vitality was robust in the absence of Tamoxifen, administration of Tamoxifen resulted in rapid cell death. FIG. 9A Immortalized NMuMG cells: in the absence (left) and presence (right) of Tamoxifen. FIG. 9B. B16 melanoma cells: in the absence (left) and presence (right) of Tamoxifen. FIG. 9C. Immortalized, Luciferase positive MEF cells transplanted into the mammary fat pad of four independent adult mice. 10 days post transplantation the mice were imaged to assess Luciferase protein levels (top). Tamoxifen was then administered to the mice, which were imaged 3 days later (bottom). FIG. 9E. Activated Caspase-3 was detectable in cells harboring the Flox-Puro^{R}/BH3 expression system within four hours of Tamoxifen treatment.

### DETAILED DESCRIPTION

Conditional gene expression systems are engineered to permit regulated expression of a gene(s) of interest. These expression systems commonly utilize the antibiotic doxycycline (i.e., “Dox systems”) to control assembly or disassembly of transcriptional activators at the gene of interest. Doxycycline control strategies have various shortcomings. For example, Dox systems rely on the assembly of numerous protein factors at a promoter site to control production of mRNA (transcription initiation) that can then be translated into the protein of interest. A duration of 4-8 hours after the introduction of doxycycline is typically needed to detect increased levels of the protein of interest in these systems (for Dox ON systems). In addition, doxycycline must be continuously present to maintain activation (or suppression, depending on the system design). Finally, doxycycline has been reported to interact with various elements of the mammalian immune system which often confounds drug treatment studies in pre-clinical oncology mouse models.

The slow expression kinetics, the need for continuous drug treatment, and the off target-effects of doxycycline are major drawbacks to the utility of the Dox system in biological research. Alternative gene expression systems, characterized by fast expression kinetics that approximate the physiology of drug treatment (where treatment can rapidly alter cellular homeostasis) and lack of reliance on the continual presence of doxycycline, will open up new possibilities for mimicking human disease in mouse models in pre-clinical settings.

Conditional gene expression systems that utilize site-specific, enzyme-mediated DNA recombination are potential alternatives (Gierut J.J., et al., Cold Spring Harb Protoc. 2014 Apr. 1; 2014(4):339-49; Sacher T., et al., Med Microbiol Immunol. 2008 June; 197(2):269-76; Garcia-Otin A. L. and Guillou F., Front Biosci. 2006 Jan. 1; 11:1108-36); for example, systems involving Cre-loxP-mediated recombination. Cre recombinase is a highly-specific DNA binding protein that efficiently recombines DNA flanked by consensus loxP site sequences. The orientation of the loxP site sequences may be designed to remove the intervening segment of DNA, and this strategy is employed ubiquitously in genetic manipulations of pre-clinical mouse models. Cre recombinase fused to the Estrogen Receptor (CreER) adds another level of control, wherein the fusion protein—in the absence of the steroid estrogen or an estrogen analog—is bound by cytoplasmic chaperones and sequestered away from the nucleus and, thus, the loxP sites. Upon binding estrogen or an estrogen analog (e.g., Tamoxifen [TMX], which unlike doxycycline is well-tolerated in animal experiments) the fusion protein undergoes a conformational change that results in its release from the cytoplasmic chaperones. The CreER fusion can then enter the nucleus and act on target loxP sequences, resulting in irreversible DNA recombination and alteration of the DNA-encoding sequence. The time period between a fusion protein's binding of estrogen or an estrogen-analog in the cytoplasm and its subsequent recombinant activity in the nucleus is short (less than 1 minute).

Various designs for controlling gene expression by enzyme-mediated recombination have been demonstrated. Many of these designs are used in transgenic mouse models and consist of an engineered construct that contains transcription suppressors, transcription terminators, polyadenylation elements, and translation suppressors, or combinations thereof. These strategies typically suppress expression (or translation) of a downstream gene (or RNA), and upon DNA recombination, the suppressive element(s) is removed and gene expression (or RNA translation) may progress. Many of these system designs comprise 3-5 kb of DNA or more.

Retroviral- and, in particular, lentiviral-based methodologies are ideal for delivering conditional gene expression systems to diverse experimental and clinical cell types. However, the nature of these methodologies inherently constrains the design of conditional gene expression systems that utilize them. For example, unlike “naked” gene delivery methods—which rely almost exclusively on biophysical properties to produce gene delivery particles—retroviral gene delivery must progress through a “life cycle” that produces infectious particles, and this life cycle limits a system's design. To be infectious, the entire viral genome must be contained within the capsid. Generally, control elements for viral production and integration comprise roughly 3 kb of genetic material, and ideally, the viral genome size should be less than 8-10 kb. This leaves approximately 5-7 kb for the addition of the genetic elements of the conditional gene expression system. Thus, large genetic elements that control gene expression may pose significant delivery problems for retroviral gene delivery.

In addition, the viral genome of retroviruses and lentiviruses is composed of RNA. Any conditional gene expression strategy that utilizes RNA processing elements (e.g., transcription suppressors, transcription terminators, or polyadenylation elements) may impact the generation of the viral RNA genome. Similarly, conditional gene expression strategies that utilize translation suppressors characterized by RNA sequences that form highly-stable, stem-loop RNA tertiary structures—which inhibit translation factors from transiting past them and, thereby, prevent the translation of downstream genes—may disrupt viral particle assembly and, thus, decrease viral production.

### Conditional Protein Translation Switches

In one aspect, the compositions of engineered conditional protein translation switches are provided. The conditional protein translation switches described herein are in the form of polynucleic acid sequences (which may also be referred to as nucleic acid molecules). As used herein, the term “polynucleic acid” refers to a string of nucleotides linked together via phosphodiester bonds. Polynucleic acids come in a variety of forms. In some embodiments, the protein translation switch is a single-stranded DNA (i.e., ssDNA). In other embodiments, the protein translation switch is a double-stranded DNA (i.e., dsDNA). In other embodiments, the protein translation switch is a single-stranded RNA (i.e., ssRNA). In yet other embodiments, the protein translation switch is a double-stranded RNA (i.e., dsRNA). In still other embodiments, the protein translation switch is a double-stranded hybrid of a ssDNA and a ssRNA.

The term “engineered,” as used herein, refers to compositions that are not found in nature. These compositions arise from human innovation. The engineered conditional protein translation switches provided herein thus typically are recombinant nucleic acid molecules.

As described herein, the engineered protein translation switches are “conditional” in that the state of the switch (i.e., translation off/suppressed or translation on/suppression relieved) is controlled by the presence of an externally controlled factor, e.g., a recombinase whose existence/abundance can be controlled or a recombinase whose activity can be induced.

In some embodiments, the engineered conditional protein translation switch comprises a polynucleic acid sequence comprising at least two Kozak translation initiation sequences together flanked by 5′ and 3′ recombination site sequences (i.e., recombinase recognition site sequences), which permit removal of the at least two Kozak translation initiation sequences from the polynucleic acid in the presence of a DNA recombinase that specifically recognizes the 5′ and 3′ recombination site sequences. In addition, the engineered conditional protein translation switch is operably linked to a sequence encoding a gene of interest, which is placed 3′ to the 3′ recombination site sequence. When operably linked in this manner, the engineered conditional protein translation switch suppresses translation of the gene of interest. Removal of the at least two Kozak translation initiation sequences from the conditional protein translation switch by DNA recombinase-mediated DNA recombination relieves the suppressed translation of the gene of interest.

The term “translation” or “protein translation” refers to a highly dynamic process wherein ribosomes “scan” the length of an mRNA to identify sites where polypeptide assembly may be initiated. The vast majority of initiation sites have the triplet codon ATG as the core sequence. The term “Kozak translation initiation sequences,” as used herein, refers to a polynucleic acid sequence that serves as a starting point for protein translation.

In some embodiments, the sequence of the at least two Kozak translation initiation sequences is RCCRCCATGG (SEQ ID NO: 1), with R being A or G. In other embodiments, the sequence of the at least two Kozak translation initiation sequences is GCCACCATGG (SEQ ID NO: 2). In some embodiments, each of the at least two Kozak translation initiation sequences is the same (e.g., GCCACCATGG [SEQ ID NO: 2]). In other embodiments, each of the at least two Kozak translation initiation sequences is unique (e.g., one sequence being GCCACCATGG [SEQ ID NO: 2] and another sequence being ACCACCATGG [SEQ ID NO: 3]). In yet other embodiments, one or more of the at least two Kozak translation initiation sequences is unique (e.g., at least one sequence being GCCACCATGG [SEQ ID NO: 2] and another sequence uniquely being ACCACCATGG [SEQ ID NO: 3]). In some embodiments, the protein translation switch has two or three Kozak translation initiation sequences.

Kozak translation initiation sequences may be separated from each other by multiple nucleotides. In some embodiments, the Kozak translation initiation sequences are separated from each other by fewer than 15 nucleotides. In some embodiments, the at least two Kozak translation initiation sequences of a protein translation switch are separated by 1, 2, or 3 nucleotides. The term “together flanked by 5′ and 3′ recombination site sequences” indicates that a recombination site sequence is positioned 5′ of the at least two Kozak translation initiation sequences and a corresponding recombination site sequence is position 3′ of the at least two Kozak translation initiation sequences (i.e., no recombination site sequence is positioned between the at least two Kozak translation initiation sequences).

As used herein, the term “recombination site sequences” refers to short polynucleic acid sequences, typically palindromic, that are specifically recognized and acted upon by a DNA recombinase. As used herein, the term “DNA recombinase” refers to a DNA modifying enzyme that binds, cleaves, strand exchanges, and rejoins DNA at its respective recombination sites (i.e., an enzyme capable of performing DNA recombination). Recombination sites and DNA recombinases have been well characterized in the prior art (see, e.g., Meinke G., et al., Chem. Rev. 2016 Oct. 26; 116(20):12785-820). DNA recombinase/recombination site sequence pairs include, but are not limited to, Cre/loxP, Dre/rox, VCre/VloxP, SCre/SloxP, Vika/vox, λ-int/attP, Flp/FRT, R/RRT,Kw/KwRT, Kd/KdRT,B2/B2RT, and B3/B3RT. In some embodiments, the recombination site sequences of a protein translation switch are selected from the list consisting of loxP (e.g., ATAACTTCGTATAATGTATGCTATACGAAGTTAT [SEQ ID NO: 4]), rox (e.g., TAACTTTAAATAATGCCAATTATTTAAAGTTA [SEQ ID NO: 5]), VloxP (e.g., TCAATTTCTGAGAATGACAGTTCTCGGAAATTGA [SEQ ID NO: 6]), SloxP (e.g., CTCGTGTCCGATAACTGTAATTATCGGACATGAT [SEQ ID NO: 7]), vox (e.g., AATAGGTCTGAGAATGGGCGTTCTCAGACGTATT [SEQ ID NO: 8]), attP (e.g., CAGCTTTTTTATACTAAGTTG [SEQ ID NO: 9]), FRT (e.g., GAAGTTCCTATACTTTCTAGAGAATAGGAACTTC [SEQ ID NO: 10]), RRT (e.g., TTGATGAAAGAATAACGTATTCTTTCATCAA [SEQ ID NO: 11]), KwRT (e.g., ACGAAAAATGGTAAGGAATAGACCATTCCTTACCATTTTTCGT [SEQ ID NO: 12]), KdRT (e.g., AAACGATATCAGACATTTGTCTGATAATGCTTCATTATCAGACAAATGTCTGATA TCGTTT [SEQ ID NO: 13]), B2RT (e.g., GAGTTTCATTAAGGAATAACTAATTCCCTAATGAAACTC [SEQ ID NO: 14]), and B3RT (e.g., GGTTCGTTAAGAATAAGTAATTCTTAAGCAACC [SEQ ID NO: 15]) site sequences. In other embodiments, the recombination site sequences are loxP site sequences. In some embodiments, the protein translation switch consists of a polynucleic acid sequence that is fewer than 500 nucleotides long. In some embodiments, the sequence is between 62 and 110 nucleotides long. In other embodiments, the protein translation switch consists of a polynucleic acid sequence that is between 62 and 150 nucleotides long. In other embodiments the protein translation switch is between 150 and 500 nucleotides long. As used herein, the term “gene of interest” refers to protein coding gene whose translation is controlled by the protein translation switch. The terms “linking” or “linked” refer to an interaction meditated by a covalent bond (e.g., in the case of a polynucleic acid the terms refer to an interaction mediated by a phosphodiester bond). To facilitate suppression of protein translation, the protein translation switch and the 3′ gene of interest are linked in such a way that the ATGs of the at least two Kozak translation initiation sequences are out of translation reading frame with the initiator ATG codon of the 3′ gene of interest. In some embodiments, the ATGs of each of the at least two Kozak translation initiation sequence are in the same translation reading frame. In other embodiments, not all of the ATGs of the at least two Kozak translation initiation sequence are in the same translation reading frame.

The term “operably linked” refers to a situation in which the linking of the protein translation switch to the sequence of a 3′ gene of interest suppresses translation of the gene of interest, and the removal of the at least two Kozak translation initiation sequences from the protein translation switch by DNA recombinase-mediated DNA recombination relieves the suppressed translation of the 3′ gene of interest.

The term “suppresses translation” refers to a decrease in the translation levels of the mRNA of a gene of interest by at least 25%, 30%, 40%, 50%, 60%, 70%, 75%, 80%, 90%, 95% or 100% relative to translation levels of the mRNA when unlinked to the protein translation switch. As used herein, the term “relieves the suppressed translation” refers to an increase in the translation levels of the mRNA of a gene of interest by at least 25%, 30%, 40%, 50%, 60%, 70%, 75%, 80%, 90%, 95% or 100% relative to translation levels of the mRNA when linked to the protein translation switch (i.e., that has not undergone DNA recombinase-mediated DNA recombination to remove the at least two Kozak translation initiation sequences). Methods of measuring and comparing translation levels are known to those skilled in the art.

### Conditional Gene Expression Systems

In another aspect, engineered conditional gene expression systems that are compatible with retroviral and lentiviral gene delivery and compositions that include such engineered conditional gene expression systems are provided. The engineered conditional gene expression systems overcome the limitations of Dox expression systems as described elsewhere herein. The term “engineered,” as used herein, refers to compositions that are not found in nature. These compositions arise from human innovation. The engineered conditional protein translation switches provided herein thus typically are recombinant nucleic acid molecules.

As described herein, the gene expression systems are “conditional” in that the state of the system (i.e., whether the system is such that the translation of an mRNA produced from a gene of interest is off/suppressed or whether the system is such that the translation of an mRNA produced from a gene of interest is on/the suppressed translation is relieved) is controlled by the presence of an externally controlled factor (e.g., a recombinase whose existence/abundance can be controlled or a recombinase whose activity can be induced).

In some embodiments, a conditional gene expression system comprises at least one polynucleic acid sequence encoded on one or more polynucleotides. As used herein, the term “polynucleic acid” refers to a string of nucleotides linked together via phosphodiester bonds. In some embodiments the conditional gene expression system is encoded on a single polynucleotide. In other embodiments, the conditional gene expression system is encoded on multiple polynucleotides.

In some embodiments, at least one polynucleotide sequence of a conditional gene expression system is in the form of single-stranded DNA (i.e., ssDNA). In other embodiments, at least one polynucleotide sequence is in the form of double-stranded DNA (i.e., dsDNA). In other embodiments, at least one polynucleotide sequence is in the form of single-stranded RNA (i.e., ssRNA). In yet other embodiments, at least one polynucleotide sequence is in the form of double-stranded RNA (i.e., dsRNA). In still other embodiments, at least one polynucleotide sequence is in the form of a double-stranded hybrid of a ssDNA and a ssRNA.

In some embodiments, the engineered conditional gene expression system comprises at least one polynucleic acid that encodes at least one DNA recombinase and at least one conditional protein translation switch operably linked to a polynucleotide sequence encoding a gene of interest. Each conditional protein translation switch comprises a polynucleotide sequence comprising at least two Kozak translation initiation sequences together flanked by 5′ and 3′ recombination site sequences (i.e., recombination recognition site sequences), which permit removal of the at least two Kozak translation initiation sequences from the polynucleic acid in the presence of a DNA recombinase that specifically recognizes the 5′ and 3′ recombination site sequences. In addition, each conditional protein translation switch is operably linked to the polynucleotide sequence encoding the gene of interest that is placed 3′ to the 3′ recombination site sequence. Furthermore, the orientation of the at least one conditional protein translation switch relative to the gene of interest is such that the polynucleotide sequence encoding the gene of interest is out of translation reading frame with the at least two Kozak translation initiation sequences of the at least one conditional protein translation switch.

In some embodiments, a conditional gene expression system comprises more than one protein translation switch, wherein each protein translation switch is operably linked to a polynucleotide sequence encoding a gene of interest, which may be the same sequence, or different sequences. For example, for different sequences, each of the two or more protein translation switches can be operably linked to a polynucleotide sequence encoding a different gene of interest or encoding a different variant of the same gene of interest.

In some embodiments, a conditional gene expression system further comprises at least one protein coding gene sequence, wherein the protein coding gene sequence is positioned 3′ of the 5′ recombination site sequence and 5′ of the at least two Kozak translation initiation sequences of a conditional protein translation switch, and DNA recombinase-mediated DNA recombination at the recombination sites removes the protein coding gene sequences and the at least two Kozak translation initiation sequences from the conditional protein translation switch. Such embodiments also can be applied to the conditional protein translation switches described above.

For example, in some embodiments, a conditional gene expression system may comprise a conditional protein translation switch, the sequence of which from 5′ to 3′ encodes: 1) a 5′ recombination site sequence; 2) a protein coding gene sequence; 3) at least two Kozak translation initiation sequences; and 4) a 3′ recombination site sequence. DNA recombinase-mediated DNA recombination of this conditional protein translation would result in the generation of a polynucleotide whose sequence retains a single recombination site sequence and lacks the protein coding gene sequence and the at least two Kozak translation initiation sequences.

In some embodiments, a conditional gene expression system comprises multiple protein translation switches each of which controls the expression of at least one 3′ gene of interest, wherein the sequence of each protein translation switch encodes, from 5′ to 3′: 1) a 5′ recombination site sequence; 2) a protein coding gene sequence; 3) at least two Kozak translation initiation sequences; and 4) a 3′ recombination site sequence. In some embodiments in which a conditional gene expression system comprises multiple protein translation switches each of which comprises a protein coding gene sequence, the protein coding gene sequence of the multiple protein translation switches is the same. In other embodiments, one or more of the protein coding gene sequences differ. For example, a conditional gene expression system may comprise one conditional protein translation switch whose sequence from 5′ to 3′ encodes: 1) a 5′ recombination site sequence; 2) a protein “A” coding gene sequence; 3) at least two Kozak translation initiation sequences; and 4) a 3′ recombination site sequence; and a second protein translation switch whose sequence from 5′ to 3′ encodes: 1) a 5′ recombination site sequence; 2) a protein “B” coding gene sequence; 3) at least two Kozak translation initiation sequences; and 4) a 3′ recombination site sequence. Preferably, in embodiments characterized by conditional gene expression systems comprising multiple protein translation switches, the recombination site sequences of the switches differ. For example, in some embodiments a conditional gene expression system comprises two protein translation switches, one comprising loxP recombination site sequences and the other comprising FRT recombination site sequences.

In the embodiments described above in which a protein coding sequence is positioned 3′ to the 5′ recombination site sequence (e.g., protein “A” as described above), the protein coding sequence can code for a marker protein, such as a selectable marker protein or a detectable marker protein. For example, a selectable marker is suitable for use in the identification of cells which have or have not been transformed or transfected with the vector. Markers include, for example, genes encoding proteins which increase or decrease either resistance or sensitivity to antibiotics or other compounds, and genes which encode enzymes whose activities are detectable by standard assays known in the art (e.g., galactosidase, fluorescence, luciferase or alkaline phosphatase). In some embodiments, the polypeptide is an enzyme that converts a substrate into a detectable molecule. In some embodiments, the reporter polypeptide comprises a luciferase enzyme, such as firefly luciferase, click-beetle luciferase, Renilla luciferase or luciferase from Oplophorus gracilirostris, such as NANOLUC®. Markers also include genes encoding proteins and genes which visibly affect the phenotype of transformed or transfected cells, hosts, colonies or plaques (e.g., a fluorescent polypeptide). Fluorescent polypeptides include, without limitation, cyan fluorescent protein (e.g., AmCyan1), green fluorescent protein (e.g., EGFP, AcGFP1, and ZsGreen1), yellow fluorescent protein (e.g., ZsYellow1 and mBanana), orange fluorescent protein (e.g., mOrange and mOrange2), red fluorescent protein (e.g., DsRed, tdTomato, mStrawberry and mCherry), and far-red fluorescent protein (e.g., HcRed1, mRaspberry and mPlum).

In other embodiments in which a protein coding sequence is positioned 3′ to the 5′ recombination site sequence (e.g., protein “A” as described above), the protein coding sequence can encode for a protein whose levels have been altered in the expressing cell (e.g., by RNAi knockdown or CRISPR-based gene inactivation). In this way, the expression system may be used to replace a gene whose expression has been experimentally manipulated. In these embodiments, the protein translation switch(es) may control the expression of a similar protein (e.g., protein “A*” as the 3′ gene of interest) that is modified in a specific way(s) as to mimic pharmacologic treatment, such as a kinase-active site mutant to mimic treatment with a kinase inhibitor.

As used herein, the term “DNA recombinase” refers to a DNA modifying enzyme that binds, cleaves, strand exchanges, and rejoins DNA at its respective recombination sites (i.e., an enzyme capable of performing DNA recombination). Various DNA recombinases have been identified in the art and include, but are not limited to, Cre, Dre, VCre, SCre, Vika, λ-Int, Flp, R, Kw, Kd, B2, and B3.

In some embodiments, the DNA recombinase is selected from the group consisting of Cre, Dre, VCre, SCre, Vika, λ-Int, Flp, R, Kw, Kd, B2, and B3, and functional variants thereof. As used herein, the term “functional variant” includes polypeptides which are about 70% identical, at least about 80% identical, at least about 90% identical, at least about 95% identical, at least about 98% identical, at least about 99% identical, at least about 99.5% identical, or at least about 99.9% identical to a protein's native amino acid sequence (i.e., wild-type amino acid sequence) and which retain recombinase functionality. The term “functional variants” also includes polypeptides which are shorter or longer than a protein's native amino acid sequence by about 5 amino acids, by about 10 amino acids, by about 15 amino acids, by about 20 amino acids, by about 30 amino acids, by about 40 amino acids, by about 50 amino acids, by about 75 amino acids, by about 100 amino acids or more and which retain recombinase functionality. The term “functional variants” also includes fusion proteins which retain recombinase functionality. In the context of a DNA recombinase variant, the term “retain recombinase functionality” refers to a variant's ability to recombine DNA at least about 5%, 10%, 20%, 25%, 30%, 40%, 50%, 60%, 70%, 75%, 80%, 90%, 100%, or more than 100% as efficiently as the respective non-variant (i.e., wild-type) DNA recombinase. Methods of measuring and comparing the efficiency of DNA recombination are known to those skilled in the art.

In some embodiments, the DNA recombinase is an inducible DNA recombinase. The term “inducible DNA recombinase” refers to a DNA recombinase whose enzymatic activity can be conditionally controlled by the presence of an inducer. Numerous inducible DNA recombinases have been described in the prior art. See e.g., Meinke G., et al., Chem. Rev. 2016 Oct. 26; 116(20):12785-820, the entire contents of which are incorporated herein by reference. For example, CreER and CreERT2 are inducible variants of Cre whose cullular localization (and, thus, enzymatic activity) can be controlled by estrogen and estrogen analogs (e.g., Tamoxifen, 4-Hydroxytamoxifen, etc.). In some embodiments, the at least one inducible DNA recombinase is selected from the list comprising CreER and CreERT2.

In some embodiments, each of the at least one DNA recombinases encoded for by the at least one polynucleic acid of the conditional gene expression system is the same, for example, CreER. In other embodiments, the at least one inducible DNA recombinases differ, for example, a conditional gene expression system may encode for both CreER and CreERT2.

As used herein, the term “recombination site sequences” refers to short polynucleic acid sequences, typically palindromic, that are recognized and acted upon by a DNA recombinase. Recombination site sequences and DNA recombinases have been well characterized in the prior art (see, e.g., Meinke G., et al., Chem. Rev. 2016 Oct. 26; 116(20):12785-820). DNA recombinase/recombination site sequence pairs include, but are not limited to, Cre/loxP, Dre/rox, VCre/ VloxP, SCre/SloxP, Vika/vox, λ-Int/attP, Flp/FRT, R/RRT, Kw/KwRT, Kd/KdRT, B2/B2RT, or B3/B3RT.

In some embodiments, the recombination site sequences of a conditional protein translation switch are selected from the group consisting of loxP (e.g., ATAACTTCGTATAATGTATGCTATACGAAGTTAT [SEQ ID NO: 4]), rox (e.g., TAACTTTAAATAATGCCAATTATTTAAAGTTA [SEQ ID NO: 5]), VloxP (e.g., TCAATTTCTGAGAATGACAGTTCTCGGAAATTGA [SEQ ID NO: 6]), SloxP (e.g., CTCGTGTCCGATAACTGTAATTATCGGACATGAT [SEQ ID NO: 7]), vox (e.g., AATAGGTCTGAGAATGGGCGTTCTCAGACGTATT [SEQ ID NO: 8]), attP (e.g., CAGCTTTTTTATACTAAGTTG [SEQ ID NO: 9]), FRT (e.g., GAAGTTCCTATACTTTCTAGAGAATAGGAACTTC [SEQ ID NO: 10]), RRT (e.g., TTGATGAAAGAATAACGTATTCTTTCATCAA [SEQ ID NO: 11]), KwRT (e.g., ACGAAAAATGGTAAGGAATAGACCATTCCTTACCATTTTTCGT [SEQ ID NO: 12]), KdRT (e.g., AAACGATATCAGACATTTGTCTGATAATGCTTCATTATCAGACAAATGTCTGATA TCGTTT [SEQ ID NO: 13]), B2RT (e.g., GAGTTTCATTAAGGAATAACTAATTCCCTAATGAAACTC [SEQ ID NO: 14]), and B3RT (e.g., GGTTCGTTAAGAATAAGTAATTCTTAAGCAACC [SEQ ID NO: 15]) site sequences.

In some embodiments, at least one conditional protein translation switch of a conditional gene expression system has recombination site sequences selected from the group consisting of loxP, rox, VloxP, SloxP, vox, attP, FRT, RRT, KwRT, KdRT, B2RT, and B3RT site sequences. In some embodiments, the recombination sites of each of the at least one protein translation switches of a conditional gene expression system are all the same sequences, for example, loxP site sequences. In other embodiments, the restriction site sequences of the at least one protein switches vary, for example, one protein translation switch may have loxP site sequences and another protein translation switch may have FRT site sequences.

In some embodiments, at least one of the at least two Kozak translation initiation sequences of at least one of the protein translation switches of each conditional protein translation switch consists of the sequence RCCRCCATGG (SEQ ID NO: 1), with R being A or G. In some embodiments, each Kozak initiation sequence in a conditional gene expression system consists of the sequence RCCRCCATGG (SEQ ID NO: 1), with R being A or G.

In some embodiments, at least one of the at least two Kozak translation initiation sequences of at least one of the protein translation switches of a conditional gene expression system consists of the sequence GCCACCATGG (SEQ ID NO: 2). In other embodiments, the sequence of each Kozak translation initiation sequence of each conditional protein translation switch is GCCACCATGG (SEQ ID NO: 2).

In some embodiments, the sequence of each of the at least two Kozak translation initiation sequences is the same. In other embodiments, the sequence of each of the at least two Kozak translation initiation sequences is unique (e.g., one sequence being GCCACCATGG [SEQ ID NO: 2] and another sequence being ACCACCATGG [SEQ ID NO: 3]). In yet other embodiments, one or more of the at least two Kozak translation initiation sequences is unique (e.g., at least sequence being GCCACCATGG [SEQ ID NO: 2] and another sequence uniquely being ACCACCATGG [SEQ ID NO: 3]).

In some embodiments, each of the protein translation switches comprises a polynucleotide sequence comprising two or three Kozak translation initiation sequences.

Kozak translation initiation sequences may be separated from each other by multiple nucleotides. In some embodiments, the at least two Kozak translation initiation sequences of a protein translation switch are separated from each other by fewer than 15 nucleotides. In some embodiments, the at least two Kozak translation initiation sequences of a protein translation switch are separated by 1, 2, or 3 nucleotides. The term “together flanked by 5′ and 3′ recombination site sequences” indicates that a recombination site sequence is positioned 5′ of the at least two Kozak translation initiation sequences and a corresponding recombination site sequence is position 3′ of the at least two Kozak translation initiation sequences (i.e., no recombination site sequence is positioned between the at least two Kozak translation initiation sequences).

As used herein, the term “gene of interest” refers to protein coding gene whose translation is controlled by a protein translation switch. The terms “linking” or “linked” refer to an interaction meditated by a covalent bond (e.g., in the case of a polynucleic acid the terms refer to an interaction mediated by a phosphodiester bond). To facilitate suppression of protein translation, the protein translation switch and the 3′ gene of interest are linked in such a ways as the ATGs of the at least two Kozak translation initiation sequences are out of translation reading frame with the initiator ATG of the 3′ gene of interest. In some embodiments, the ATGs of each of the at least two Kozak translation initiation sequence are in the same translation reading frame. In other embodiments, not all of the ATGs of the at least two Kozak translation initiation sequence are in the same translation reading frame.

The term “operably linked” refers to a situation in which the linking of the protein translation switch to the sequence of a 3′ gene of interest suppresses translation of the gene of interest, and the removal of the at least two Kozak translation initiation sequences from the protein translation switch by DNA recombinase-mediated DNA recombination relieves the suppressed translation of the 3′ gene of interest.

The term “suppresses translation” refers to a decrease in the translation levels of an mRNA of a gene of interest by at least 25%, 30%, 40%, 50%, 60%, 70%, 75%, 80%, 90%, 95% or 100% relative to translation levels of the mRNA of the gene of interest when unlinked to the protein translation switch. As used herein, the term “relieves the suppressed translation” refer to an increase in the translation levels of an mRNA of a gene of interest by at least 25%, 30%, 40%, 50%, 60%, 70%, 75%, 80%, 90%, 95% or 100% relative to translation levels of the mRNA of the gene of interest when linked to the protein translation switch (i.e., that has not undergone DNA recombinase-mediated DNA recombination to remove the at least two Kozak translation initiation sequences). Methods of measuring and comparing translation levels are known to those skilled in the art.

In some embodiments a conditional gene expression system comprises a single polynucleic acid sequence that encodes for the at least one DNA recombinase and the at least one protein translation switch linked to at least one gene of interest. In some embodiments, the single polynucleic acid sequences is less than 7 kb in length. In other embodiments, the sequence is less than 6 kb in length. In still other embodiments, the sequence is less than 5 kb in length. In yet other embodiments, the sequences is less than 4, 3, or 2 kb in length.

In some embodiments a conditional gene expression system comprises two polynucleic acid sequences: one that encodes for the at least one DNA recombinase and one that encodes for the at least one protein translation switch linked to at least one gene of interest. In some embodiments, the polynucleic acid sequence encoding for the protein translation switch (with is accompanying linked gene of interest) is less than 5 kb in length. In other embodiments, the single nucleic acid sequence is less than 3 kb in length. In still other embodiments, the sequence is less than 2 kb in length. In other embodiments, the sequence is less than 1 kb in length.

In some embodiment, the conditional gene expression system is amenable to adenoviral or lentiviral delivery. The term “delivery” refers to the introduction of the conditional gene expression system into a biological sample (e.g., a cell). In some embodiments, the conditional gene expression system, in its entirety, can be packaged within an adenoviral or lentiviral capsid. In some embodiments, the components of the conditional gene expression system are packaged within multiple adenoviral or lentiviral capsids. For example, in some embodiments, a polynucleotide encoding for a DNA recombinase and a polynucleotide encoding for a protein translation switch are incorporated into separate viral genomes and packaged into separate viral capsids.

In some aspects, the disclosure relates to viral genomes comprising a conditional gene expression system as described above. In some embodiments, the viral genome is an adenovirus genome or a lentivirus genome.

In other aspects, the disclosure relates to engineered virions comprising a conditional gene expression system as described above. The term “virion,” as used herein, refers to a complete viral particle constituting the infective form of a virus. In some embodiments, the engineered virion is a lentivirus virion or an adenovirus virion.

### Methods of Mimicking Clinical Pharmacology and Validating Potential Drug Targets

The disclosed conditional protein translation switches and conditional gene expression systems can be used in a variety of methods. For example, the ability to switch from no protein expression to protein expression using the conditional protein translation switches and conditional gene expression systems allows for investigating the effects of a protein controlled by the switch or system. Such methods are useful, in some aspects, in mimicking certain physiological effects and for testing therapeutics.

For example, disclosed herein are methods of mimicking clinical pharmacology in a pre-clinical setting. Previously described pre-clinical models are poor substitutes for clinical settings. The conditional gene expression systems described herein are superior in that they can keep genetic perturbation silent until such time as a researcher chooses to induce translation of the gene of interest, more closely mimicking the clinical setting. These systems can also facilitate the validation of potential drug target efficacy before ever developing a drug. For example, these systems can be used to validate target hits identified in gene deletion screens (e.g., CRISPR and RNAi).

In some aspects, also provided herein are methods of testing the effect of a product of a gene of interest comprising introducing an engineered conditional gene expression system, a recombinant viral genome comprising an engineered conditional gene expression system, or a virion comprising a conditional gene expression system into a biological sample, a cell or a test animal, expressing the at least one DNA recombinase or inducing the at least one inducible DNA recombinase to initiate DNA recombination, and analyzing the impact of DNA recombination relative to a control. In some embodiments, the gene of interest is identified by a gene deletion screen, such as but not limited to RNAi or CRISPR-based screening procedures. In some embodiments, the cell is a tumor cell.

The term “conditional gene expression system” as used herein refers to any conditional gene expression system described above in the section “Conditional Gene Expression Systems.” As used herein, the term “control” refers to a biological sample, cell, or test animal: 1) lacking the conditional gene expression system; 2) containing only components of the conditional gene expression system (e.g., lacks a recombinase); or 3) containing the entire conditional expression system, but lacking the induction necessary for DNA-recombinase mediated DNA recombination (e.g., lacks the presence of Tamoxifen).

As used herein, the term “introduction” refers to any mechanism whereby polynucleic acid molecules can be incorporated into a living biological sample, cell, or animal Examples include, but are not limited to, transduction, transfection (e.g., DEAE dextran-mediated transfection, CaPO₄-mediated transfection, lipid-mediated DNA uptake, and PEI-mediated DNA uptake, laser transfection), transformation (e.g., calcium chloride, electroporation, and heat-shock), gene transfer, and particle bombardment. In some embodiments, the conditional gene expression system is introduced into biological samples or cells in vitro. In other embodiments the conditional gene expression system is introduced into a test animal in vivo.

### EXAMPLES

### Example 1

### Conditional Gene Expression System Design

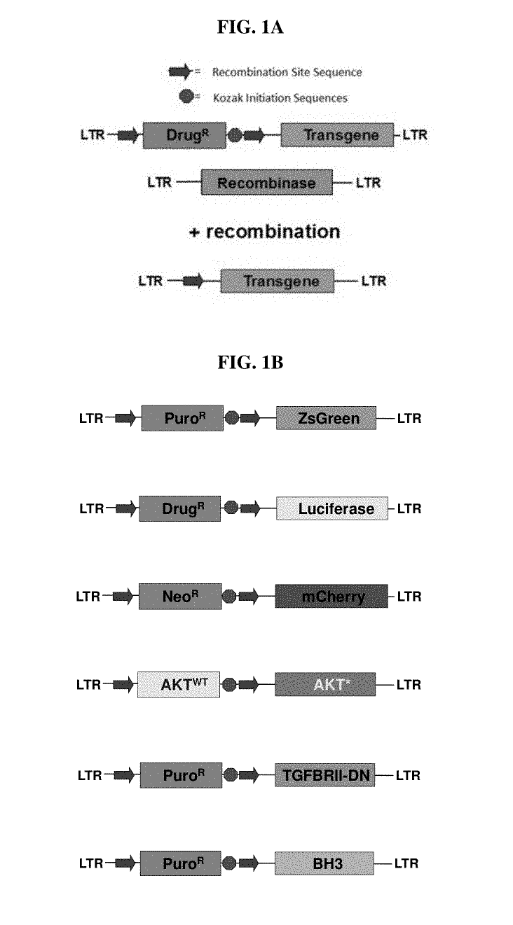

Multiple conditional gene expression systems were designed following the same general structure: at least one polynucleic acid that encodes for 1) a protein translation switch linked to a 3′ gene of interest and 2) a DNA recombinase (FIG. 1A). The design of the protein translation switches also followed a general structure: at least two Kozak translation initiation sequences of the sequence RCCRCCATGG (SEQ ID NO: 1) (with R being A or G) flanked by loxP recombination site sequences (i.e., Floxed). An optional gene-coding segment was typically included downstream of the first loxP sequence and upstream of the protein translation switch (e.g., a selective drug marker) (FIG. 1A).

Importantly, the ATGs of the Kozak translation initiation sequences are out of frame with the ATG start codon of the gene of interest. Kozak sequences are thought to help ensure fidelity of translation initiation at a gene's initiator ATG rather than ATG sites present in the interior of the gene contained in the mRNA. Because the mRNA expressed from the polynucleic acid that encodes for a protein translation switch upstream of a 3′ gene of interest contains multiple ATG sites, translation may initiate at any of these sites by different ribosomes. The protein translation switch achieves translation suppression by recruiting RNA-bound ribosomes and initiating translation distal to and out-of-frame with the initiator ATG of the gene of interest.

The expression system design allows for removal of the Kozak initiation sequences from the translation suppressor switch (and, if included, the optional gene-coding segment) via enzyme-mediated, site-specific DNA recombination (FIG. 1A). This recombination results in the proper translation of the gene(s) of interest—causing an increase in the gene's protein levels.

Various conditional gene expression systems were designed using this general structure. Polynucleic acid components of these conditional gene expression systems included Flox-Puro^{R}/ZsGreen, which facilitates conditional translation of ZsGreen; Flox-Drug^{R}/Luciferase, which facilitates conditional translation of Luciferase; Flox-Neo^{R}/mCherry, which facilitates conditional translation of mCherry; Flox-AKT^{Wt}/AKT*, which facilitates conditional translation of AKT*, a kinase-dead AKT; Flox-Puro^{R}/TGBRII-DN, which facilitates conditional translation of TGFBRII-DN (dominant negative TGF-β Receptor II), a cytoplasmic truncation that blocks downstream signaling after binding of TGF-β; and Flox-Puro^{R}/BH3, which facilitates conditional translation of BH3, a mitochondria-based inducer of apoptosis (FIG. 1B).

### Example 2

### Design of a Highly Efficient, Short Translation Suppressor Switch

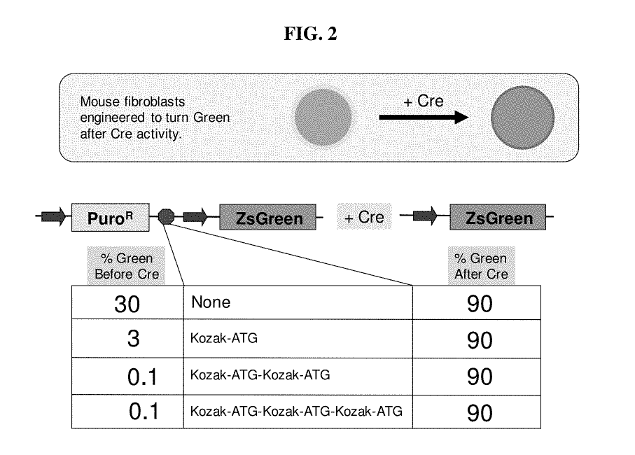

The translation suppressor switch impacts the ability of ribosomes to reinitiate protein translation at the ATG start site of the gene of interest after scanning/passing through the switch. Experiments were performed to determine the efficiency of the switches and how the presence of multiple Kozak translation initiation sequences in the translation suppressor switch impacts the switch's efficiency. Flox-Puro^{R}/ZsGreen conditional gene expression systems were generated in which the number of Kozak initiation sequences of the translation suppressor switches varied (e.g., no Kozak initiation sequence, one Kozak initiation sequence, two Kozak initiation sequences, and three Kozak initiation sequences). The efficiency of the various translation suppressor switches was then assessed in MEF cells harboring the conditional gene expression systems (FIG. 2). These conditional gene expression systems, which included Cre recombinase, were delivered to the cells via lentiviral delivery. These studies demonstrated that translation suppressor switches characterized by two or three Kozak initiation sequences were 300 and 30 times more efficient at repressing the translation of ZsGreen relative to those that lacked a Kozak initiation sequence and to those characterized by one Kozak initiation sequence, respectively. Indeed, only 0.1% of MEF cells harboring a Flox-Puro^{R}/ZsGreen conditional gene expression system that was characterized by a translator suppressor switch with two or three Kozak initiation sequences were ZsGreen positive in the absence of Tamoxifen.

### Example 3

### Efficiency of the Conditional Gene Expression Systems In Vitro and In Vivo

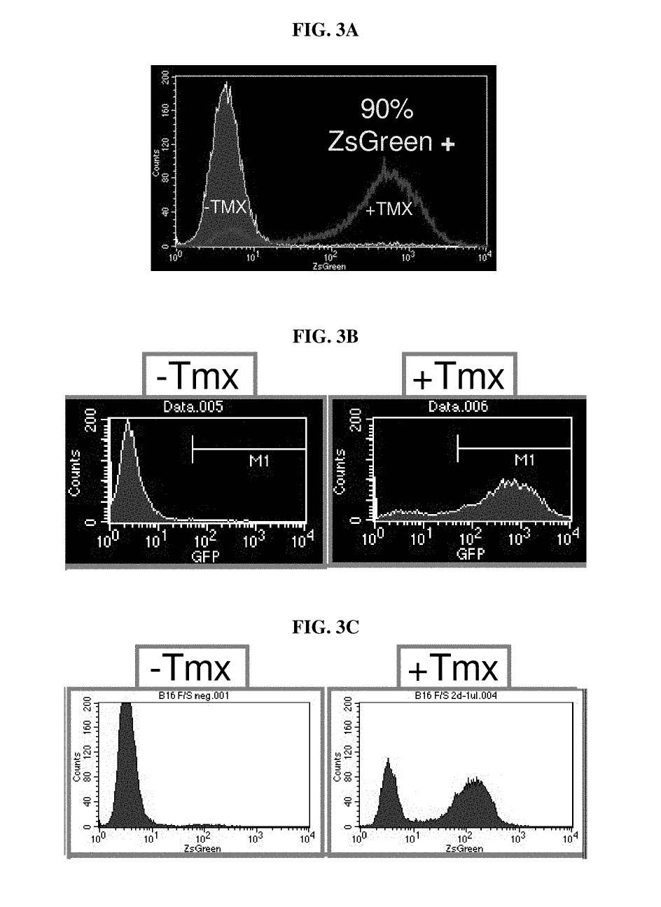

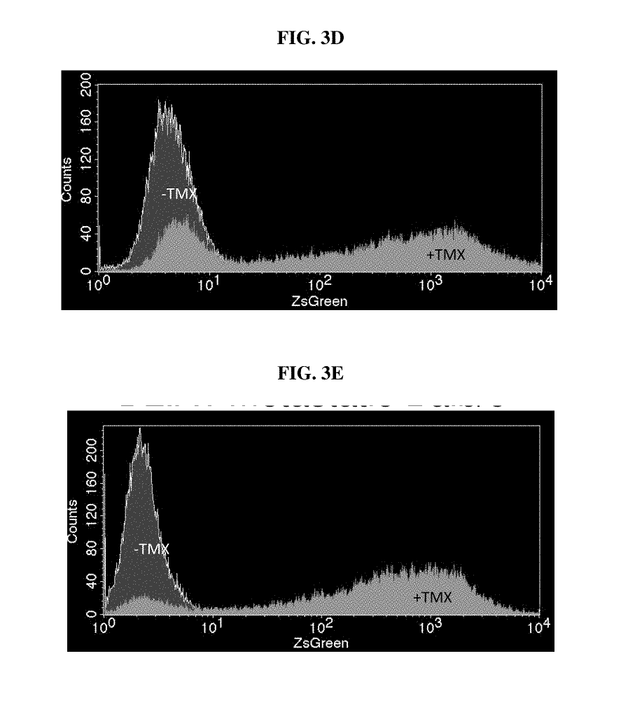

The efficiency of the conditional gene expression systems was assessed in vitro in various cell types. Unlike the analyses performed above (i.e., described in FIG. 2), here, the conditional gene expression systems that were delivered to the cells via lentiviral delivery included CreER instead of Cre. The conditional gene expression systems functioned efficiently in all of the cell types analyzed (FIGS. 3A-3E) and remained efficient in cells harboring multiple conditional gene expression systems (FIGS. 4A-4B).

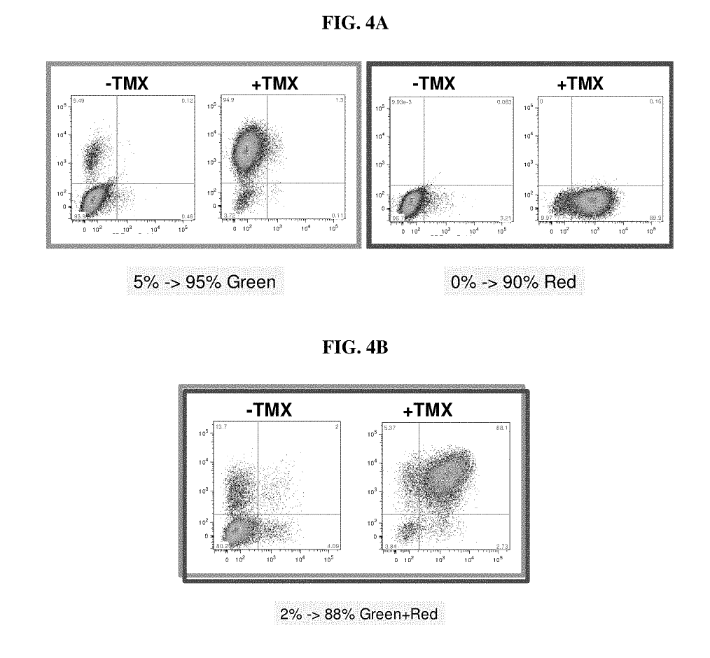

Experiments were then performed to determine the efficiency of the conditional gene expression systems in vivo. Here, Lewis lung carcinoma cells (FIG. 5A) or primary mouse fibroblasts (FIG. 5B) harboring the Flox-Puro^{R}/Luciferase conditional gene expression system or 4T1 carcinoma cells harboring the Flox-Puro^{R}/ZsGreen conditional gene expression system were subcutaneously transplanted into the mammary fat pad of female adult mice (FIG. 5D). The efficiencies of the conditional gene expression systems were assessed in the presence and absence of tamoxifen. In each instance, the protein levels of the gene of interest were largely undetectable in the absence of Tamoxifen; however, after exposure to Tamoxifen, robust protein levels of the gene of interest were evident. Thus, the conditional gene expression systems function efficiently in vivo.

### Example 4

### Kinetics of the Conditional Gene Expression Systems

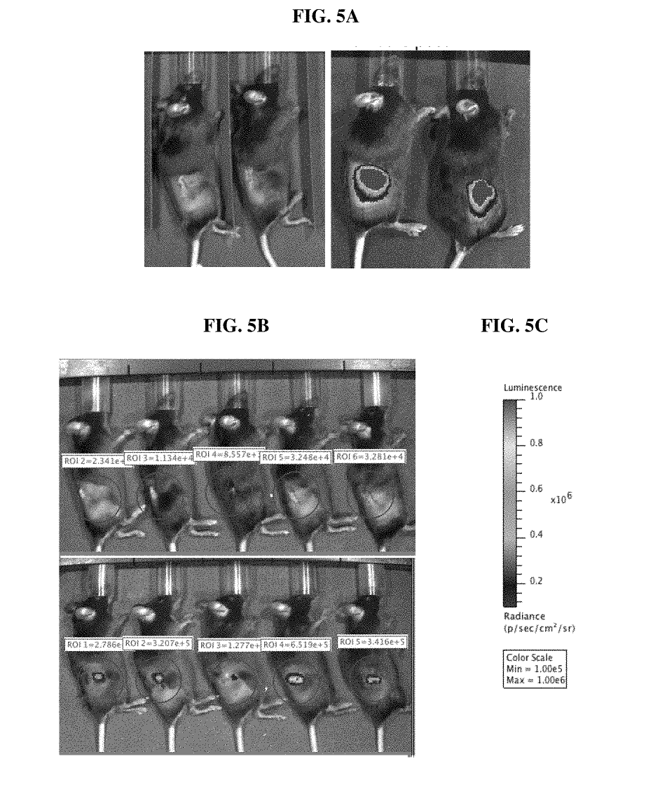

Experiments were performed to determine the responsiveness of the conditional gene expression systems. First, the responsiveness of DNA recombination was assessed. Here, cells harboring the Puro^{R}/ZsGreen conditional gene expression system were exposed to Tamoxifen for various durations of time. Following cellular exposure to Tamoxifen, the cells were washed to remove any residual Tamoxifen. Induction of ZsGreen protein levels were assessed 48 hours later by FACs sorting (FIG. 6A). Recombination of the Puro^{R}/ZsGreen polynucleic acid sequence was seen with 1 minute of exposure to Tamoxifen.

Next, the responsiveness of protein translation was assessed. Here, the induction of Luciferase protein levels was determined in cells harboring the Flox-Puro^{R}/Luciferase conditional gene expression system. Luciferase protein levels were determined at various time points following exposure to Tamoxifen (FIG. 6B). Induction was detectable within three hours following administration of Tamoxifen.

### Example 5

### Mimicking Clinical Pharmacology in a Pre-Clinical Setting

The conditional gene expression systems described herein can serve as valuable tools in early-stage drug research and development, particularly in oncology screening and target validation. The protein translation switch can control the expression of a gene(s) of interest in established tumors, allowing researchers to more effectively model the “real world” dynamics of clinical pharmacology in pre-clinical models. For example, existing pre-clinical models manipulate gene expression before implanting tumors in mice, so the tumor is genetically and phenotypically altered from the beginning. This approach is a poor substitute for clinical settings where a patient presents an established tumor and is treated acutely with a cancer therapy. In pre-clinical models, the protein translation switch of the conditional gene expression systems described herein can keep the genetic perturbation silent until such time as the researcher chooses to induce translation of the gene of interest via administration of estrogen or an estrogen analog, more closely mimicking the clinical setting. Importantly, before recombination-mediated expression of the gene-of-interest, the conditional expression system may be configured to express a gene product whose sequence is placed within the protein translation switch (i.e., 3′ of the 5′ recombination site sequence and 5′ of the Kozak initiation sequences).

The context may be a large-scale screen of hundreds of genes of interest in a single tumor to identify biologically relevant genes or a small analysis of a single gene of interest to validate the role of a particular gene in the pre-clinical model. To demonstrate the feasibility of these applications of the conditional gene expression systems described herein, three proof of principle experiments were performed.

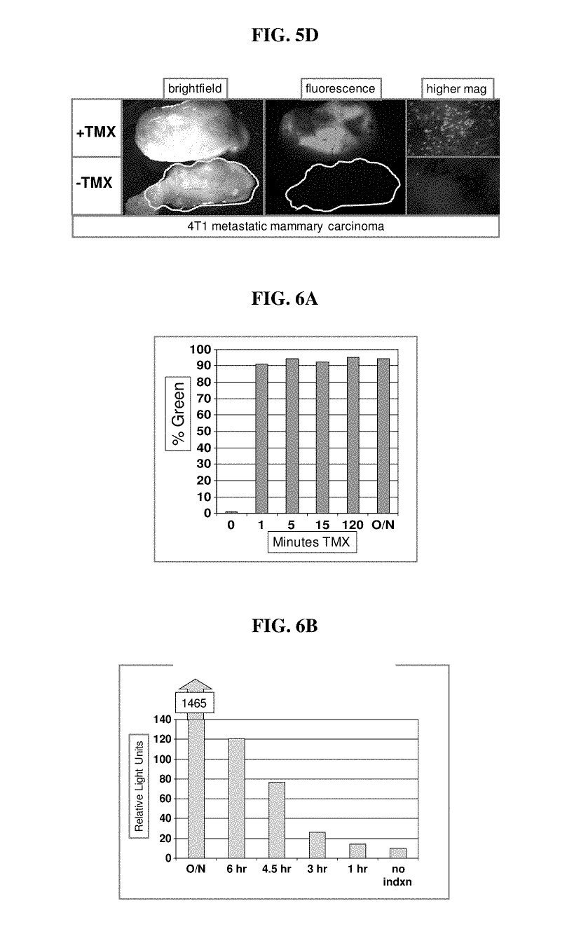

First, experiments were designed using the Flox-AKT^{Wt}/AKT* conditional gene expression system to mimic clinical kinase inhibitor treatment in the pre-clinical setting (FIG. 7). miRNA-mediated endogenous AKT knockdown was performed in MCF10A cells harboring the Flox-AKT^{Wt}/AKT* conditional gene expression system. Cell harboring the expression system were selected, plated, and grown for 2 days. Cell growth was assessed thereafter after brief (4 hour) treatment with Tamoxifen (resulting in the production of AKT*, an active-site mutant of AKT kinase) or no treatment (resulting in the maintenance of production of wild type AKT). Conditional expression of the active-site mutant AKT kinase mimicked AKT kinase inhibitor treatment (FIG. 7).

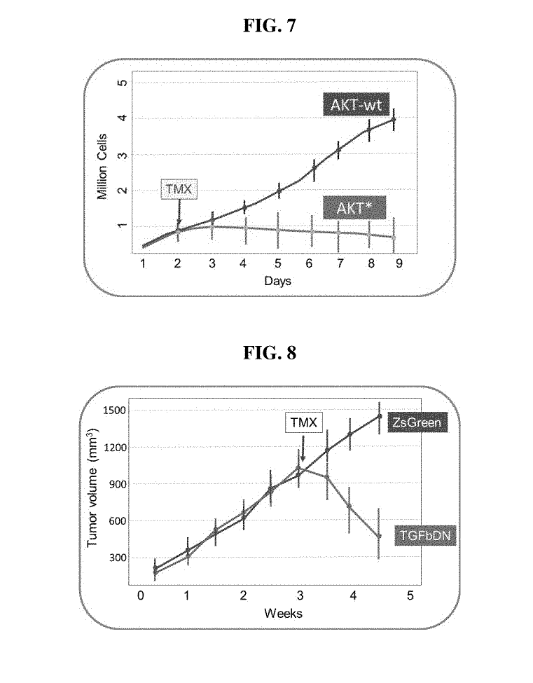

Second, experiments were designed using the Flox-Puro^{R}/TGFBRII-DN conditional gene expression system to model the blockade of TGF-β signaling in tumors in vivo (FIG. 8). TGFBRII-DN (dominant negative TGF-β Receptor II) is a cytoplasmic truncation that blocks downstream signaling after binding of TGF-β. Blockade of TGF-β causes rapid tumor cell death. 4T1 carcinoma cells harboring the Flox-Puro^{R}/TGFBRII-DN conditional gene expression system or the Flox-Puro^{R}/ZsGreen conditional gene expression system were subcutaneously transplanted into adult mice and grown for 3 weeks. Tumor size was assessed after administration of Tamoxifen. Induction of TGFBRII-DN caused rapid tumor cell death (FIG. 8).

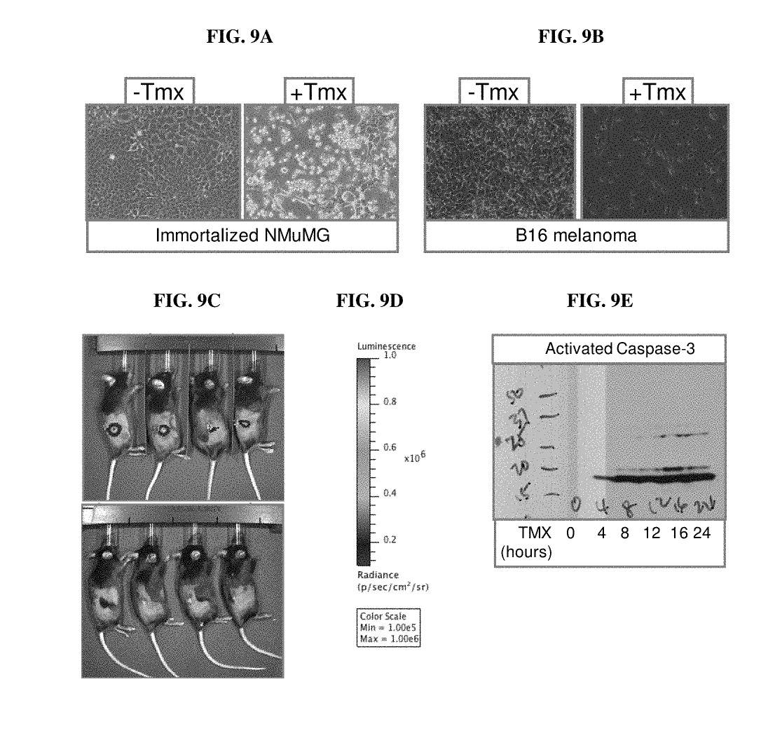

Third, experiments were designed to model cytotoxic chemotherapy therapy using the Flox-Puro^{R}/BH3 conditional gene expression system. This system facilitates conditional translation of BH3 which drives apoptosis through mitochondrial disruption. Various cell lines harboring this system underwent apoptosis after exposure to Tamoxifen in vitro (FIGS. 9A-9B). Similar results were seen in vivo (FIG. 9C-9D). Activated Caspase-3 was detectable in cells harboring the Flox-Puro^{R}/BH3 expression system within four hours of Tamoxifen treatment, indicating activation of the core apoptosis components (FIG. 9E). This rapid activation of Caspase-3 is similar to other apoptosis inducers such as UV, FasL, and poisons. Thus, the Flox-Puro^{R}/BH3 conditional gene expression system exhibits tight control of a killer molecule and facilitates rapid conditional induction.

Thus, the conditional gene expression systems described herein facilitate the mimicking of clinical pharmacology in a pre-clinical setting. These systems can also facilitate the validation of potential drug target efficiency before ever developing a drug. For example, these systems can be used to validate target hits identified in gene deletion screens (e.g., CRISPR and RNAi).

### REFERENCES

1. Garcia-Otin A. L. and Guillou F., Mammalian genome targeting using site-specific recombinases, Front. Biosci., 2006. 11: p. 1108-36.
2. Gierut J. J., Jacks T. E., and Haigis K. M., Strategies to achieve conditional gene mutation in mice, Cold Spring Harb. Protoc., 2014. 2014(4): p. 339-49.
3. Meinke G., Bohm A., Hauber J., Pisabarro M. T., and Buchholz F., Cre Recombinase and Other Tyrosine Recombinases, Chem. Rev., 2016. 116(20): p. 12785-820.
4. Sacher T., Jordan S., Mohr C. A., Vidy A., Weyn A. M., Ruszics Z., and Koszinowski U. H., Conditional gene expression systems to study herpesvirus biology in vivo, Med. Microbiol. Immunol., 2008. 197(2): p. 269-76.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

1. An engineered conditional protein translation switch comprising a polynucleic acid sequence comprising at least two Kozak translation initiation sequences together flanked by 5′ and 3′ recombination site sequences, which permit removal of the at least two Kozak translation initiation sequences from the polynucleic acid in the presence of a DNA recombinase that specifically recognizes the 5′ and 3′ recombination site sequences, and wherein:
(a) linking the protein translation switch to a sequence encoding a gene of interest placed 3′ to the 3′ recombination site sequence suppresses translation of the gene of interest, and
(b) the removal of the at least two Kozak translation initiation sequences from the conditional protein translation switch by DNA recombinase-mediated DNA recombination relieves the suppressed translation of the gene of interest, and
optionally wherein the at least two Kozak translation initiation sequences are positioned 1, 2, or 3 nucleotides from each other.

2. The engineered conditional protein translation switch of claim 1, wherein the polynucleic acid sequence of the conditional protein translation switch comprises two or three Kozak translation initiation sequences together flanked by the 5′ and 3′ recombination site sequences.

3. (canceled)

4. The engineered conditional protein translation switch of claim 1, wherein the sequence of each of the at least two Kozak translation initiation sequences is:
(a) RCCRCCATGG (SEQ ID NO: 1), with R being A or
(b) GCCACCATGG (SEQ ID NO: 2).

5. (canceled)

6. The engineered conditional protein translation switch of claim 1, wherein the recombination site sequences are selected from the group consisting of loxP, rox, VloxP, SloxP, vox, attP, FRT, RRT, KwRT, KdRT, B2RT, and B3RT site sequences.

7. The engineered conditional protein translation switch of claim 1, wherein the recombination site sequences are loxP site sequences.

8. An engineered conditional gene expression system comprising at least one polynucleic acid wherein the at least one polynucleic acid encodes at least one DNA recombinase and at least one conditional protein translation switch operably linked to a polynucleotide sequence encoding a gene of interest, wherein:
(a) each conditional protein translation switch comprises a polynucleotide sequence comprising at least two Kozak translation initiation sequences together flanked by 5′ and 3′ recombination site sequences which permit removal of the at least two Kozak translation initiation sequences from the polynucleic acid in the presence of a DNA recombinase that specifically recognizes the 5′ and 3′ recombination site sequences, and
(b) each conditional protein translation switch is operably linked to the polynucleotide sequence encoding the gene of interest that is placed 3′ to the 3′ recombination site sequence, wherein the orientation of the at least one conditional protein translation switch relative to the gene of interest is such that the polynucleotide sequence encoding the gene of interest is out of translation reading frame with the at least two Kozak translation initiation sequences of the at least one conditional protein translation switch, and
optionally wherein the at least two Kozak translation initiation sequences of each conditional protein translation switch are positioned 1, 2, or 3 nucleotides from each other.

9. The engineered conditional gene expression system of claim 8, wherein the engineered conditional gene expression system comprises more than one protein translation switch, wherein each protein translation switch is operably linked to a polynucleotide sequence encoding a gene of interest, optionally wherein:
(a) the polynucleotide sequences encoding each gene of interest are different sequences; and/or
(b) each protein translation switch comprises unique recombination site sequences.

10. (canceled)

11. The engineered conditional gene expression system of claim 8, further comprising at least one protein-coding gene sequence, wherein:
(a) each protein-coding gene sequence is positioned 3′ of the 5′ recombination site sequence and 5′ of the at least two Kozak translation initiation sequences of a conditional protein translation switch, and
(b) DNA recombinase-mediated DNA recombination at the recombination sites removes the protein-coding gene sequence and the at least two Kozak translation initiation sequences from the conditional protein translation switch, and
optionally wherein each protein-coding sequence is uniquely positioned 3′ of the 5′ recombination site sequence and 5′ of the at least two Kozak translation initiation sequences of a conditional protein translation switch.

12. (canceled)

13. The engineered conditional gene expression system of claim 8, wherein the at least one DNA recombinase is selected from the group consisting of Cre, Dre, VCre, SCre, Vika, λ-Int, Flp, R, Kw, Kd, B2, B3, and functional variants thereof.

14. The engineered conditional gene expression system of claim 8, wherein the at least one DNA recombinase is an inducible DNA recombinase optionally wherein the inducible recombinase is selected from the group consisting of CreER and CreERT2.

15. (canceled)

16. The engineered conditional gene expression system of claim 8, wherein each conditional protein translation switch comprises a polynucleotide sequence comprising two or three Kozak translation initiation sequences.

17. (canceled)

18. The engineered conditional gene expression system of claim 8, 17, wherein:
(a) the sequence of at least one of the at least two Kozak translation initiation sequences of at least one conditional protein translation switch is RCCRCCATGG (SEQ ID NO: 1), wherein R is A or G;
(b) the sequence of each Kozak translation initiation sequence of each conditional protein translation switch is RCCRCCATGG (SEQ ID NO: 1), wherein R is A or G;
(c) the sequence of at least one Kozak translation initiation sequence of at least one conditional protein translation switch is GCCACCATGG (SEQ ID NO: 2); or
(d) the sequence of each Kozak translation initiation sequence of each conditional protein translation switch is GCCACCATGG (SEQ ID NO: 2).

19.-21. (canceled)

22. The engineered conditional gene expression system of claim 8, wherein:
(a) the recombination site sequences of at least one conditional protein translation switch is selected from the group consisting of loxP, rox, VloxP, SloxP, vox, attP, FRT, RRT, KwRT, KdRT, B2RT, and B3RT site sequences;
(b) the recombination site sequences of each conditional protein translation switch is selected from the group consisting of loxP, rox, VloxP, SloxP, vox, attP, FRT, RRT, KwRT, KdRT, B2RT, and B3RT site sequences; or
(c) the recombination site sequences of each conditional protein translation switches is a loxP site sequence.

23.-24. (canceled)

25. The engineered conditional gene expression system of claim 8, wherein a single polynucleic acid encodes for the at least one DNA recombinase and the at least one conditional protein translation switch.

26. A recombinant viral genome comprising the conditional gene expression system of claim 8, optionally wherein the viral genome is an adenovirus genome or a lentivirus genome.

27. (canceled)

28. An engineered virion comprising the conditional gene expression system of claim 8, optionally wherein the virion is an adenovirus or a lentivirus virion.

29. (canceled)

30. An engineered virion comprising the recombinant viral genome of claim 26.

31. A method of testing the effect of a product of a gene of interest comprising:
(a) introducing the engineered conditional gene expression system of claim 8 into a biological sample, a cell or a test animal,
(b) expressing the at least one DNA recombinase or inducing the at least one inducible DNA recombinase to initiate DNA recombination, and
(c) analyzing the impact of DNA recombination relative to a control.

32. The method of claim 31, wherein the gene of interest is identified by a gene deletion screen.

33. The method of claim 31, wherein the cell is a tumor cell.
